(19) United States
(12) Patent Application Publication
(10) Pub. No.: US 2024/0417345 A1
(43) Pub. Date: Dec. 19, 2024

(54) PROCESS FOR ISOMERIZATION OF 2,4,4-TRIMETHYLPENT-2-ENE TO 2,4,4-TRIMETHYLPENT-1-ENE

(71) Applicant: Evonik Oxeno GmbH & Co. KG, Marl (DE)

(72) Inventors: Peter Kucmierczyk, Herne (DE); Robert Franke, Marl (DE); Anna Chiara Sale, Duesseldorf (DE); Dirk Fridag, Haltern am See (DE); Ana Markovic, Haltern am See (DE); Armin Matthias Rix, Marl (DE); Maren Termühlen, Dortmund (DE); Laura-Selin Harding, Dorsten (DE); Jan Benedikt Metternich, Recklinghausen (DE)

(73) Assignee: Evonik Oxeno GmbH & Co. KG, Marl (DE)

(21) Appl. No.: 18/738,261

(22) Filed: Jun. 10, 2024

(30) Foreign Application Priority Data
Jun. 14, 2023 (EP) 23179200.3

(51) Int. Cl. C07C 5/25; B01J 29/70; B01J 31/08

(52) U.S. Cl. CPC C07C 5/2518; B01J 29/7007; B01J 31/08; C07C 5/2568; B01J 2231/52; C07C 2529/70; C07C 2531/10

(57) ABSTRACT

A process isomerizes 2,4,4-trimethylpent-2-ene to 2,4,4-trimethylpent-1-ene over a heterogeneous catalyst based on a zeolite or an ion-exchange resin. The process includes providing an input stream containing 2,4,4-trimethylpent-2-ene and 2,4,4-trimethylpent-1-ene and performing the isomerization with the input stream using a heterogeneous catalyst based on a zeolite or an ion-exchange resin. A proportion of 2,4,4-trimethylpent-2-ene in the total stream is at least 25 mol % based on the total input stream.

### Cross-Reference to Related Applications

This patent application is based on and claims priority pursuant to 35 U.S.C. § 119(a) to European Patent Application No. 23179200.3, filed on Jun. 14, 2023, in the European Patent Office, the entire disclosure of which is hereby incorporated by reference herein.

### BACKGROUND

### Field of the Invention

The present invention relates to a process for isomerization of 2,4,4-trimethylpent-2-ene to 2,4,4-trimethylpent-1-ene over a heterogeneous catalyst based on a zeolite or an ion-exchange resin.

### Description of Related Art

Diisobutene is an industrially relevant product obtained by dimerization of isobutene. Diisobutene consists of the isomers 2,4,4-trimethylpent-1-ene (hereinbelow also: TMP1) and 2,4,4-trimethylpent-2-ene (hereinbelow also: TMP2) with a mass distribution of TMP1:TMP2 of about 78:22 to 81:19 (equilibrium distribution). This mixture can be converted into higher-value products inter alia in carbonylation processes. Especially in carbonylation processes, such as methoxycarbonylation (reaction product here is methyl 3,5,5-trimethylhexanoate) or hydroformylation (reaction product here is 3,5,5-trimethylhexanal), the internal olefin TMP2 exhibits markedly lower reactivity than the terminal olefin TMP1. As a consequence of inadequate stability of the catalysts or economic factors (for example the size of the reactor), it is often not possible to adjust the reaction conditions or residence times in these carbonylation reactions sufficiently to allow the terminal TMP2 to react to completion.

A direct use of the unreacted TMP2 is disadvantageous for reasons of reactivity. It is therefore advantageous to convert the TMP2 or a portion thereof into the more reactive form TMP1 by isomerization in order ultimately to allow conversion into valuable products.

Processes for isomerization of TMP2 to TMP1 are known in the literature. However, these processes are performed under homogeneous catalysis and based on strong mineral acids such as for example H₂SO4, H₃PO₄ or benzenesulfonic acid (cf. U.S. Pat. No. 2,554,251 A). However, homogeneously catalyzed reaction systems have the disadvantage that the isomerization catalysts must be removed again from the obtained pure TMP1 or the obtained TMP1/TMP2 mixtures in a costly and inconvenient manner. Only then could they be supplied to the aforementioned carbonylation processes. Acidic systems are generally also corrosive, thus requiring high-quality materials.

### SUMMARY OF THE INVENTION

It is accordingly an object of the present invention to provide a process for isomerization of 2,4,4-trimethylpent-2-ene to 2,4,4-trimethylpent-1-ene which does not exhibit the aforementioned problems. However, the process should also be able to achieve high conversions.

This object was achieved by the process of the invention described herein. Preferred embodiments are also specified.

According to the invention the process is a process for isomerization of 2,4,4-trimethylpent-2-ene to 2,4,4-trimethylpent-1-ene, wherein the process comprises at least the following steps:
a. providing an input stream, containing 2,4,4-trimethylpent-2-ene and 2,4,4-trimethylpent-1-ene, the proportion of 2,4,4-trimethylpent-2-ene in the total stream being at least 25 mol %, based on the total input stream;
b. performing the isomerization with the input stream using a heterogeneous catalyst based on a zeolite or an ion-exchange resin,
wherein the proportion of 2,4,4-trimethylpent-2-ene in the product stream is lower and the proportion of 2,4,4-trimethylpent-1-ene in the product stream is higher than in the input stream.

The advantage of the process according to the invention is the use of a heterogeneous catalyst system. Such a catalyst system need not be removed from the product stream but remains in the reaction vessel/the reactor. The catalyst systems according to the invention based on a zeolite or on an ion-exchange resin also allow high conversions to be achieved.

### DETAILED DESCRIPTION OF THE INVENTION

The input stream to be employed contains 2,4,4-trimethylpent-2-ene and 2,4,4-trimethylpent-1-ene, the proportion of 2,4,4-trimethylpent-2-ene in the total stream being at least 25 mol %, preferably at least 28 mol %, based on the total input stream. Streams having less trimethylpent-2-ene are so close to the equilibrium distribution that use is not worthwhile. Such streams may be diisobutene streams producible by dimerization from isobutene or isobutene-containing hydrocarbon mixtures as disclosed in EP 1 360 160 B1 for example. In addition, the streams to be employed here may be obtained as unreacted residual streams in carbonylation processes, for example in methoxycarbonylation or in hydroformylation.

The isomerization in step b. may in principle be performed in any suitable reactor. It is possible for the isomerization to take place in a single reactor or in two or more reactors connected in parallel or in series. Performance in batches or in continuous operation is also possible. The isomerization is preferably carried out in one or more continuously operated reactors that are customarily employed in solid/liquid contact reactions. When using continuously operated flow reactors, a fixed bed is usually, but not always, employed. When a fixed-bed flow reactor is used, the liquid can flow in an upward or downward direction. In most cases, downward flow of the liquid is preferable. In addition, it is possible to operate the reactor with product recycling or in straight pass. A concept distinct from fixed bed reactors is represented for example by reactors in which the ion exchanger or zeolite is suspended in a liquid phase.

Reactors employed in the isomerization according to the invention are preferably tubular reactors or tube bundle reactors, in particular those having internal tube diameters of 10 to 60 mm. The length-to-diameter ratio of the catalyst bed may be varied here, either by the geometric dimensions of the reactor or by its fill level. At the same contact amount and load (LHSV), it is thus possible to achieve different empty-tube velocities.

The heating of the tubes of the reactor, whether it be a tubular reactor or a tube bundle reactor, can be effected by means of steam or heat carrier oils via the reactor shell. Especially when using liquid heating media, the shell side is constructed such that the temperature gradient in contact with all tubes is as homogeneous as possible. The technical measures necessary for this are known to those skilled in the art and are described in the literature (installation of baffle plates, disc-on-donut construction, infeed/outfeed of heat-transfer medium at various points in the reactor, etc.). Preferably, the reaction medium and heat transfer medium are respectively conveyed through the reactor tubes and reactor shell in cocurrent flow, more preferably from top to bottom. A preferred embodiment is described for example in DE 10 2006 040 433 A1.

The isomerization of 2,4,4-trimethylpent-2-ene to 2,4,4-trimethylpent-1-ene takes place exothermically, i.e. it proceeds with the release of energy, which results in warming of the reaction mixture. The temperature rise may be limited by diluting the input stream, for example by recycling product.

The reactor(s) used in the isomerization may be operated adiabatically, polytropically or practically isothermally. Practically isothermally means that the temperature is at no point in the reactor more than 10° C. higher than the temperature at the reactor entrance. In the case of adiabatic operation of the reactors, it is usually advantageous to arrange a plurality of reactors in series and to provide cooling between the reactors. Reactors that are suitable for polytropic or practically isothermal operation are for example the tube bundle reactors already mentioned and also stirred-tank reactors and loop reactors.

The process can be executed at rather mild temperatures. The isomerization in step b is preferably performed at a temperature of 25° C. to 90° C., preferably 30° C. to 80° C., particularly preferably 35° C. to 70° C.

In addition, the isomerization of the invention can be carried out at a pressure equal to or greater than the vapour pressure of the input stream mixture and/or of the reaction mixture at the respective reaction temperature, preferably at a pressure of less than 40 bar.

The isomerization in step b. is preferably also performed in the liquid phase. It should be clear that in this case the pressure and temperature must be chosen such that the input stream is present, or may be present, in the liquid phase.

In the isomerization according to the invention, the reactor(s) may also contain two or more catalysts based on a zeolite or an ion-exchange resin. For example, a mixture of ion-exchange resins of different reactivity may be used. It is likewise possible for a reactor to contain catalysts of different activities that are arranged for example in layers. If more than one reactor is used, the individual reactors may be filled with the same or different catalyst(s) based on a zeolite or on an ion-exchange resin.

The isomerization according to the invention employs a catalyst based on a zeolite or an ion-exchange resin as the heterogeneous catalyst. Appropriate zeolites and ion-exchange resins are widely available and known to those skilled in the art. It has been found that the catalyst based on a zeolite preferably has a Si:Al ratio in the range from 40:1 to 200:1. In the case of catalysts based on an ion-exchange resin, preferably the styrene-divinylbenzene type as the H-form and in a partially neutralized form has in addition been found to be highly suitable.

When the catalyst is a zeolite, some zeolites have proven to be particularly advantageous, for example beta-and gamma-zeolites. The zeolite is therefore preferably selected from the group consisting of Z-beta-H-25, Z-beta-H-38, Z-beta-H-360, Z-Mor-H-20, Z-Y-H-60, Z-Y-H-80, Z-beta-H, Z-CFG-1, Z-beta-ammonium-38, Z-CBV 760 CY (1.6), Z-CBV 780 CY (1.6), CP 814E CY (1.6), CBV 500 CY (1.6), H-CZB-150 and mixtures thereof.

The ion-exchange resin employed may be for example ion-exchange resins produced by sulfonation of phenol/aldehyde condensates or by sulfonation of co-oligomers of aromatic vinyl compounds. Examples of aromatic vinyl compounds for the production of the co-oligomers are: styrene, vinyltoluene, vinylnaphthalene, vinylethylbenzene, methylstyrene, vinylchlorobenzene, vinylxylene and divinylbenzene. Particular preference is given to using for the isomerization ion-exchange resins produced by the sulfonation of co-oligomers formed by the reaction of styrene with divinylbenzene. The ion-exchange resins may be produced in gel-like, macroporous or sponge-like form. The properties of these resins, in particular specific surface area, porosity, stability, swelling or shrinkage and exchange capacity may, as is known, be varied via the production process.

lon-exchange resins of the preferred styrene-divinylbenzene type are sold inter alia under the following trade names: CT 151 and CT275 from Purolite, Amberlyst® 15, Amberlyst® 35, Amberlite® IR-120, Amberlite® 200 from Rohm&Haas, Dowex M-31 from Dow, Lewatit® K 2621, Lewatit® K 2431 from Lanxess.

The pore volume of the ion-exchange resins employable as catalysts, in particular those of the preferred styrene-divinylbenzene type, is preferably 0.3 to 0.9 ml/g, more preferably 0.5 to 0.9 ml/g. The pore volume can be determined for example by adsorptive techniques. The particle size of the ion-exchange resins is preferably from 0.3 mm to 1.5 mm, more preferably 0.5 mm to 1.0 mm. A narrower or broader particle size distribution may be chosen. It is thus possible for example to use ion-exchange resins having a very uniform particle size (monodisperse resins).

The ion-exchange resins employable as catalysts for the isomerization may be present as partially neutralized ion-exchange resins. For this purpose, the ion-exchange resin may be treated with acids or bases, as described in EP 1 360 160 B1.

The isomerization in step b. then affords a product stream having a proportion of the 2,4,4-trimethylpent-2-ene which is lower and a proportion of the 2,4,4-trimethylpent-1-ene in the product stream which is higher than in the input stream. In a preferred embodiment of the present invention, the proportion of 2,4,4-trimethylpent-1-ene in the product stream is from 60 to 80 mol %, preferably 65 to 80 mol %. The proportion of 2,4,4-trimethylpent-2-ene in the product stream is then 20 to 40 mol %, preferably 20 to 35 mol %.

The invention is described hereinbelow by reference to examples. These are provided for elucidation purposes and do not limit the subject matter of the invention.

### EXAMPLES

### Example 1

2.4 g of a catalyst according to the invention (Purolite CT 275, partially neutralized, 0.9 eq/L) were weighed into a tubular reactor having a diameter of 0.6 cm. The reactor was supplied with 2,4,4-trimethylpent-2-ene (>99%). The isomerization was carried out at a temperature of 55° C. and ambient pressure. After exiting the reactor the distribution of 2,4,4-trimethylpent-2-ene to 2,4,4-trimethylpent-1-ene was determined (in the input stream 2,4,4-trimethylpent-2-ene: 2,4,4-trimethylpent-1-ene 99:1). Analysis was by gas chromatography. Peak areas were evaluated by the external calibration method.

| | | w fraction of | w fraction of |
|---|---|---|---|
| Mass stream of | | 2,4,4-trimethylpent-2-ene | 2,4,4-trimethylpent-1-ene |
| 2,4,4-trimethylpent-2-ene | Temperature | in diisobutene isomers | in diisobutene isomers |
| [g/min] | [° C.] | in product stream [%] | in product stream [%] |
| 0.1 | 55 | 22^{a} | 78^{a} |

^{a}Over experimental duration of 80 hours.

The results show that the catalyst according to the invention is very suitable for the isomerization.

### Example 2

2.0 g of a catalyst according to the invention (CBV760) were weighed into a tubular reactor having a diameter of 0.6 cm. The reactor was supplied with 2,4,4-trimethylpent-2-ene (>99%). The 2,4,4-trimethylpent-2-ene was passed through the reactor at a volume flow of 0.12 g/min. The isomerization was carried out at a temperature of 55° C. and ambient pressure. After exiting the reactor the distribution of 2,4,4-trimethylpent-2-ene to 2,4,4-trimethylpent-1-ene was determined (in the input stream 2,4,4-trimethylpent-2-ene:2,4,4-trimethylpent-1-ene 99:1). Analysis was by gas chromatography. Peak areas were evaluated by the external calibration method.

| | | w fraction of | w fraction of |
|---|---|---|---|
| Mass stream of | | 2,4,4-trimethylpent-2-ene | 2,4,4-trimethylpent-1-ene |
| 2,4,4-trimethylpent-2-ene | Temperature | in diisobutene isomers | in diisobutene isomers |
| [g/min] | [° C.] | in product stream [%] | in product stream [%] |
| 0.126 | 55 | 27^{a} | 73^{a} |

^{a}Over experimental duration of 24 hours.

The results show that the catalyst according to the invention is very suitable for the isomerization.

1. A process for isomerization of 2,4,4-trimethylpent-2-ene to 2,4,4-trimethylpent-1-ene, wherein the process comprises:
a. providing an input stream, comprising 2,4,4-trimethylpent-2-ene and 2,4,4-trimethylpent-1-ene, a proportion of 2,4,4-trimethylpent-2-ene in the total stream being at least 25 mol %, based on the total input stream;
b. performing the isomerization with the input stream using a heterogeneous catalyst based on a zeolite or an ion-exchange resin,
wherein a proportion of 2,4,4-trimethylpent-2-ene in a product stream is lower than in the input stream and a proportion of 2,4,4-trimethylpent-1-ene in the product stream is higher than in the input stream.

2. The process according to claim 1, wherein the proportion of 2,4,4-trimethylpent-1-ene in the product stream is from 60 to 80 mol %.

3. The process according to claim 1, wherein the proportion of 2,4,4-trimethylpent-2-ene in the product stream is from 20 to 40 mol %.

4. The process according to claim 1, wherein the isomerization in b is performed at a pressure of less than 40 bar.

5. The process according to claim 1, wherein the isomerization in b is performed at a temperature of 25° C. to 90° C.

6. The process according to claim 1, wherein the zeolite has a Si: Al ratio in a range from 40:1 to 200:1.

7. The process according to claim 1, wherein the zeolite is selected from the group consisting of beta-and gamma-zeolites.

8. The process according to claim 1, wherein the catalyst is a zeolite selected from the group consisting of Z-beta-H-25, Z-beta-H-38, Z-beta-H-360, Z-Mor-H-20, Z-Y-H-60, Z-Y-H-80, Z-beta-H, Z-CFG-1, Z-beta-ammonium-38, Z-CBV 760 CY (1.6), Z-CBV 780 CY (1.6), CP 814E CY (1.6), CBV 500 CY (1.6), H-CZB-150 and mixtures thereof.

9. The process according to claim 1, wherein the ion-exchange resin employed is selected from the group consisting of ion-exchange resins produced by sulfonation of phenol/aldehyde condensates and ion-exchange resins produced by sulfonation of co-oligomers of aromatic vinyl compounds.

10. The process according to claim 1, wherein the aromatic vinyl compounds for producing the co-oligomers are selected from the group consisting of styrene, vinyltoluene, vinylnaphthalene, vinylethylbenzene, methylstyrene, vinylchlorobenzene, vinylxylene and divinylbenzene.

11. The process according to claim 1, wherein the ion-exchange resin employed is anion-exchange resin produced by sulfonation of co-oligomers formed by reaction of styrene with divinylbenzene.

12. The process according to claim 1, wherein the ion-exchange resin employed is a partially neutralized ion-exchange resin.

13. The process according to claim 1, wherein the isomerization is performed in a liquid phase.

14. The process according to claim 1, wherein the proportion of 2,4,4-trimethylpent-1-ene in the product stream is from 65 to 80 mol %.

15. The process according to claim 1, wherein the proportion of 2,4,4-trimethylpent-2-ene in the product stream is from 20 to 35 mol %.

16. The process according to claim 1, wherein the isomerization in b is performed at a temperature of 35° C. to 70° C.
